(19) United States
(12) Patent Application Publication
(10) Pub. No.: US 2013/0309676 A1
(43) Pub. Date: Nov. 21, 2013

(54) BIASED N-MERS IDENTIFICATION METHODS, PROBES AND SYSTEMS FOR TARGET AMPLIFICATION AND DETECTION

(71) Applicant: ALFRED E. MANN FOUNDATION FOR SCIENTIFIC RESEARCH, (US)

(72) Inventor: Scott Peter LAYNE, Los Angeles, CA (US)

(73) Assignee: ALFRED E. MANN FOUNDATION FOR SCIENTIFIC RESEARCH, Santa Clarita, CA (US)

(21) Appl. No.: 13/844,341

(22) Filed: Mar. 15, 2013

Related U.S. Application Data
(60) Provisional application No. 61/648,804, filed on May 18, 2012.
(60) Provisional application No. 61/668,904, filed on Jul. 6, 2012.

(51) Int. Cl. C12Q 1/68

(52) U.S. Cl. CPC C12Q 1/6858
USPC 435/6.12; 435/289.1; 536/23.1; 435/287.2

(57) ABSTRACT

A method for selecting oligonucleotides for biased polynucleotide amplification is described. Related probes, methods and systems are also described.

### CROSS REFERENCE TO RELATED APPLICATION

The present application claims priority to U.S. Provisional Application No. 61/648,804 filed on May 18, 2012 and U.S. Provisional Application No. 61/668,904 filed on Jul. 6, 2012, both of which are incorporated herein by reference in its entirety.

### FIELD

The present disclosure relates to probe sequence identification and related methods and systems, and in particular to biased N-mers identification methods, probes and systems for target amplification and detection. More particularly, the present disclosure relates to biased N-mers identification methods and systems as well as to related probes, devices, methods and systems wherein N-mers are used for target amplification possibly followed by downstream detection.

### BACKGROUND

Amplification and detection of targets and in particular amplification and detection of biomarkers has been a challenge in the field of biological molecule analysis, in particular when performed in samples wherein additional analytes interfere with the desired target detection. Whether for pathological examination or for fundamental biology studies, several methods are commonly used for the detection of various classes of biomaterials and biomolecules and in particular polynucleotide markers.

Some methods are in particular used which are based on isothermal polynucleotide amplification or Polymerase Chain Reaction (PCR) based amplification. These methods have provided the ability to amplify and detect one or more polynucleotide biomarkers in biological samples and are also suitable for diagnostic purposes.

Despite development of various methods and systems, however, achievement of target amplification and detection with a desired and in particular high sensitivity and specificity remains challenging.

### SUMMARY

Provided herein in accordance with several embodiments of the present disclosure are methods and systems for identification of biased N-mers and related oligonucleotide probes suitable to be used in methods and systems for detection of polynucleotide targets. In particular, in some embodiments, N-mers of the disclosure can be used to increase sensitivity in methods to detect polynucleotides wherein detection is performed with higher sensitivity and specificity compared to methods without biased N-mers.

According to a first aspect, a method to select N-mers of about 9 to 16 nucleotides for manufacturing oligonucleotide probes biased towards a target polynucleotide of a target organism is described. The method comprises determining frequency distribution and location of N-mers in the target polynucleotide to obtain a target N-mer distribution; determining frequency distribution of N-mers in contaminants to obtain a contaminant N-mers distribution. The method further comprises selecting a set of N-mers based on the target N-mer distribution and location, and on the contaminant N-mers distribution and location to provide a selected N-mers for the manufacturing of the oligonucleotide probes.

According to a second aspect, a method to select a set of N-mers of about 9 to 16 nucleotides for manufacturing oligonucleotide probes biased towards a target polynucleotide is described. The method comprises determining N-mers of the target polynucleotide to identify N-mers specific to the target polynucleotide; determining a frequency distribution and location of the N-mers specific to the target polynucleotide within the target polynucleotide to provide a target N-mers distribution; determining a frequency distribution of the N-mers specific to the target polynucleotide within one or more contaminants to provide a contaminant N-mers distribution; and selecting a set N-mers based on the target N-mer distribution and location, and on the contaminant N-mers distribution to provide a selected N-mers for the manufacturing of the oligonucleotide probes.

According to a third aspect, a computer-based method to select N-mers of about 9 to 16 nucleotides biased towards a target polynucleotide of a target whole organism is provided. The method comprising the following computer-operated steps wherein a computer performs the steps in single-processor mode or multiple-processor mode is described. In particular, the method comprises determining frequency distribution and location of N-mers in the target polynucleotide to obtain a target N-mer distribution; determining frequency distribution of N-mers in contaminants to obtain a contaminant N-mers distribution; and selecting a set of N-mers based on the target N-mer distribution and location, and on the contaminant N-mers distribution and location to provide a selected N-mers for the manufacturing of the oligonucleotide probes.

According to a fourth aspect, a device to perform detection of a target polynucleotide is provided. The device comprising a first component configured to perform amplification of one or more target nucleic acid from a sample to provide an amplification mixture, a second component configured to perform specific detection of one or more target nucleic acid from the amplification mixture, and an electronic interface to collect data from portable point of care device or non-portable point of care device to a computer or smart device.

The N-mers, and related probes, methods and systems herein provided, allow in several embodiments detection of one or more target polynucleotide with higher sensitivity and specificity with respect to some methods of the art.

The N-mers, and related probes, methods and systems herein provided, allow in several embodiments detection of viral, bacterial, cell, yeast, parasite and fungal sequences from single or mixed DNA and RNA viruses derived from environmental or clinical samples.

The N-mers, and related probes, methods and systems herein described can be used in connection with applications wherein detection of polynucleotide targets and/or of organism associated thereto is desired. Exemplary applications comprise diagnostics, other medical applications, fundamental biological science, diagnostics, veterinary, forensics, environmental, biosecurity, species bar coding.

The details of one or more embodiments of the disclosure are set forth in the accompanying drawings and the detailed description and examples below. Other features, objects, and advantages will be apparent from the detailed description, examples and drawings, and from the appended claims.

### BRIEF DESCRIPTION OF THE DRAWINGS

The accompanying drawings, which are incorporated into and constitute a part of this specification, illustrate one or more embodiments of the present disclosure and, together with the detailed description and the examples, serve to explain the principles and implementations of the disclosure.

FIG. 1 shows an annotated schematic representation of a genome sequence from a target organism (see 3′-5′line target organism—left panel) and a genome sequence from a contaminant organism (3′-5′ line contaminant organism right panel) with an exemplary list of few possible sequences (indicated as Sequence 1 through Sequence 9 for each organism, in FIG. 1) to be amplified in a target and in a contaminant organism In the exemplary illustration of FIG. 1, “Sequence 5” is identified as an unique or specific target sequence.

FIG. 2 shows diagrams providing an exemplary schematic representation of the results of a non-biased polynucleotide amplification of Sequences 1 to 9 of FIG. 1 for the target organism (left panel) and for contaminant organism (right panel). In the exemplary embodiment of FIG. 2, amplification comprises techniques such as Multiple Displacement Amplification (MDA), sample amplification by φ29 DNA polymerase with random hexamer primers, performed on the exemplary sample of FIG. 1. Amplification, represented as bars, shows amplification of all sequences with no one sequences favored including Sequence 5.

FIG. 3 shows diagrams providing a schematic representation of the results of a biased polynucleotide amplification of Sequences 1 to 9 of FIG. 1 for the target organism (left panel) and for the contaminant organism (right panel). In the exemplary embodiment of FIG. 3, amplification comprises techniques such as MDA, sample amplification by a polymerase, for example φ29 DNA polymerase, with N-mers primers obtainable with methods and systems herein described, performed on the exemplary sample of FIG. 1. Amplification, represented as bars, shows higher amplification of Sequence 5 in the target organism versus the contaminant organism and higher amplification than the other sequences in either organism.

FIG. 4 shows a schematic representation of an exemplary amplification of a target polynucleotide performed with probes having sequences from biased N-mers herein described. In particular, the schematic illustration shows a semi-specific, self-focusing isothermal amplification of DNA performed on a target DNA. In the exemplary illustration of FIG. 4, for self-focusing to take place, the distance between opposing primers should be no greater than the average length of single-stranded amplicons.

FIG. 5 shows a schematic representation of an exemplary amplification of contaminant polynucleotide (50) performed with probes (51) having sequences from biased N-mers herein described. In particular, the schematic illustration shows the semi-specific, self-focusing isothermal amplification of DNA (52) with the primers of FIG. 4 performed on a contaminant DNA present in the same sample of the DNA.

FIG. 6 shows a graph providing a schematic representation of the “DNA Mass” produced over time in self focusing amplification and sporadic amplification for an exemplary amplification of contaminant polynucleotide performed with probes having sequences from biased N-mers herein described. In particular, the schematic illustration shows the semi-specific, self-focusing isothermal amplification of DNA with the primers of FIG. 4 performed on a contaminant DNA present in the same sample of the DNA. In the illustration of the figure for self-focusing to take place, the distance between opposing primers should be no greater than the average length of single-stranded amplicons.

FIG. 7 shows a schematic description of an exemplary biased N-mer Amplification of DNA with Termination of a unique specific target DNA (710) and its adjacent DNA (720) according to embodiments herein described. DNA synthesis is primed by biased N-mers (primers, 730). Exponential amplification, denoted by the extending arrow (740) occurs by a “branching” mechanism. φ29 DNA polymerase acts to extend primers (N-mers, 730), as it displaces downstream DNA products. The ratio of dideoxynucleotides to deoxynucleotides (ddNTPs/NTPs) determines the statistics of chain termination as denoted by an “X” symbol (750). Lower ratios result in longer synthetic fragments. Higher ratios result in shorter synthetic fragments.

FIG. 8 shows a block diagram of a method to perform biased amplification according to embodiments herein described.

FIG. 9 shows an exemplary embodiment of the method described in FIG. 8 to perform biased amplification.

FIG. 10 shows another example embodiment of the method described in FIG. 8 to perform biased amplification.

FIG. 11 shows a computer system that can be used to implement the methods described to select N-mers.

FIG. 12 shows an exemplary selection process of N-mers.

### DETAILED DESCRIPTION

According to an embodiment of the present disclosure, methods to obtain an oligonucleotide probe sequence for amplification and detection of one or more polynucleotide targets or polynucleotides target group are provided.

The term “oligonucleotide” as used herein refers to a polynucleotide with three or more nucleotides. Nucleotides in oligonucleotides can include natural bases or modified bases, such as methylated, deaminated, and phosphothioated bases. In the present disclosure, oligonucleotides serve as “probes” and/or “primers”, when used for amplification and/or detection of one or more corresponding target polynucleotides. The term “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term “polynucleotide” includes nucleic acids of any length, and in particular DNA, complimentary DNA, RNA, messenger RNA, micro RNA, analogs and fragments thereof.

The term “target” as used herein indicates an analyte of interest. The term “analyte” refers to a substance, compound, moiety, or component whose presence or absence in a sample is to be detected. The term “sample” as used herein indicates a limited quantity of something that is indicative of a larger quantity of that something, including but not limited to fluids from a biological environment, specimen, cultures, tissues, synthetic compounds or portions thereof. In particular, “target polynucleotide” refers to a polynucleotide of interest whose presence or absence in a sample is to be detected. Exemplary target polynucleotides include polynucleotides be associated with a biological environment in which the term “biological environment” refers to any biological setting, including, for example, ecosystems, orders, families, genera, species, subspecies, organisms, tissues, cells, viruses, organelles, cellular substructures, prions, and samples of biological origin. Additional exemplary target polynucleotide comprise in particular polynucleotide that can be associated with a specific state of a biological environment including but not limited to a phase of cellular cycle, health and disease state as a biomarker of said state. The presence, absence, reduction, upregulation of the biomarker is associated with and is indicative of a particular state.

Exemplary target polynucleotides which are also biomolecules and possibly biomarkers comprise sequences associated to an organism or biological particle (e.g. a virus), in particular, genomic sequence of a target organism or particle, which includes sequences of any nuclear, mitochondrial, and plasmid DNA, as well as any other nucleic acids carried by the organism or particle which are detectable with methods and systems herein described.

In embodiments of the disclosure, presence or absence of target polynucleotides are to be detected in a sample known or expected to also include one or more contaminants. The term “contamination” as used herein relates to an unwanted constituent (contaminant) in a sample to analyzed or related biological environment. In particular, “contaminant” can be referred to a polynucleotide, a sequence and/or an organism or particle associated thereto.

The term “false positive” as defined herein indicates a result of a medical, chemical or biological test that comes out as positive but is erroneous. The term “true positive” as defined herein indicates a result of a test (e.g. a medical, chemical, or biological test) that reflects a positive result, which is an accurate positive result. The term “false negative” as defined herein indicates a result of a test a test (e.g. a medical, chemical, or biological test) that comes out as negative but is erroneous. The term “true negative” as defined herein indicates a result of a test (e.g. a medical, chemical, or biological test) that reflects a negative result, which is an accurate negative result. The term “unique sequence” as used herein indicates a unique continuous series of nucleotides, wherein the term “unique” indicates a feature that is peculiar and distinctive for a referenced item. In particular a “unique sequence” as used herein indicates a continuous series of nucleotides that is peculiar to and can be used to distinguish a referenced item associated to the sequence.

In particular, according to embodiments herein described oligonucleotides can be provided to be used as probes for amplification and detection of one or more target polynucleotide of interest, which are based on biased N-mers identified by the method of the disclosure.

The term “N-mers” as used herein indicates a string of codes specifying a pattern of a specific length of nucleotide sequence. In the present disclosure, “N” is defined as the number of nucleotides in a “N-mer”; for example, a 9-mer is a N-mer with 9 nucleotides and a 16-mer is a N-mer with 16 nucleotides. In some embodiments, and N-mer is comprised of DNA nucleotides (A, T, G, C) or RNA nucleotides (A, U, G, C). The selection of N-mer length or the number of nucleotides in an N-mer can be decided by one skilled in the art. A skilled person understands that the N-mer length can depend on the frequency that particular N-mers are found within the target sequence.

In particular, N-mers herein provided can have a length of 9 to 16 nucleotides. In accordance with embodiments of the present disclosure, N-mers can be selected in view of the properties of polymerases to be used in connection with oligonucleotides having the N-mers sequence. In particular, DNA polymerases require oligonucleotide primers to hybridize to single stranded DNA to begin double strand synthesis. Consequently, the rate at which double strand DNA synthesis begins is related to the rate at which oligonucleotide primers hybridize to single stranded DNA. Such rates of hybridization can be related to the melting temperatures of oligonucleotide as given by Tₘ=4(G+C)+2(A+T) C°. In general, this expression reveals that shorter oligonucleotides hybridize faster than longer ones and that priming events are more prevalent and/or frequent for shorter oligonucleotides than longer ones. More prevalent and/or frequent priming events result in faster target DNA amplification rates and shorter amplification times. For reactions that are desired to produce significant amplification products within minutes, oligonucleotide primers consisting of N-mers (with about 9≦N≦16) offer a practical means of achieving fast kinetics.

In particular, N-mers herein described are biased towards one or more target polynucleotides. The terms “bias” and “biased” as used herein indicates N-mers that are more frequently present within the target polynucleotide sequence and less frequently within the contaminant genome. On the contrary, “unbiased” N-mers indicate N-mers having equal probability of appearing in both the target and the contaminant DNA. Exemplary unbiased N-mers comprise N-mers that are randomly selected from the genomic sample. According to several example embodiments of the present disclosure, in DNA amplification and detection, a collection of variable length polynucleotides (N-mers) can be used as biased primers to amplify specific target DNA contained in a genomic sample. In general the sequence of an N-mer can be chemically synthesized as an oligonucleotide that can act as a primer. The genomic sample can be comprised of both the target DNA and other contaminant DNA. When primers comprising unbiased N-mers are used for amplification, all DNA in the genomic sample, such as target and contaminant DNA, are amplified. However, if the N-mers are “biased”, then the target DNA are preferentially amplified compared to the rest of the contaminant DNA. For example, consider a target DNA of length 200, and the net sequence length of all contaminant DNA to be 25000. Consequently, consider an N-mer of length 10 with the sequence “ACTGAACGTG”. If this sequence appears 40 times in the target DNA and 5000 times in the contaminant DNA, then the probability of this N-mer in both the target and contaminant DNA is the same (40/200=5000/25000=0.2). Therefore, this N-mer can be considered unbiased with respect to the given target and contaminant DNAs. However, if another N-mer of sequence “GTCGAGTCGA” appears of 80 times in the target sequence and 2000 times in the contaminant DNA, then the probability of this N-mer to be found in the target DNA is 80/200=0.4, and the probability within the contaminant DNA is 2000/25000=0.08. Therefore, this N-mer is considered to be biased towards the target DNA.

Biased N-mers, in accordance with the present disclosure, can be identified and selected by a method based on frequency determination of N-mers in a target polynucleotide and in contaminants. In accordance with the present disclosure, amplification of low quantities of DNA to detectable quantities of DNA can be performed by isothermal methods. Among the various methods, those based on priming with random hexamers and amplification by a suitable polymerase, such as Φ29 DNA polymerase, or a combination of amplifying enzymes identifiable by a skilled person in the art, can be useful. Such isothermal methodology is termed as “Multiple Displacement Amplification” and can be used to amplify all DNA within samples and can have applications to genomics, forensics and diagnostics. The use of relatively short random hexamer primers can result in faster annealing rates to complimentary DNA and faster amplification of all DNA within samples. Such amplification can proceed without bias or non-representation. For point-of-care molecular diagnostics, a skilled person can amplify all or some of the DNA contained within samples if desired. In some embodiments, a skilled person can amplify specific and relatively short DNA sequences within samples if desired. In embodiments where relatively short DNA sequences are to be amplified, a skilled person can use, for example, relatively long specific primers (usually 18-30 nucleotides) at high concentrations in combination with a polymerase such as Φ29 DNA polymerase. As previously illustrated, relatively long specific primers (even at high concentrations) can reduce DNA amplification rates, since longer primers can have higher melting temperatures, Tₘ=4(G+C) 2(A+T) C, and/or slower annealing rates and/or efficiencies to complimentary DNA.

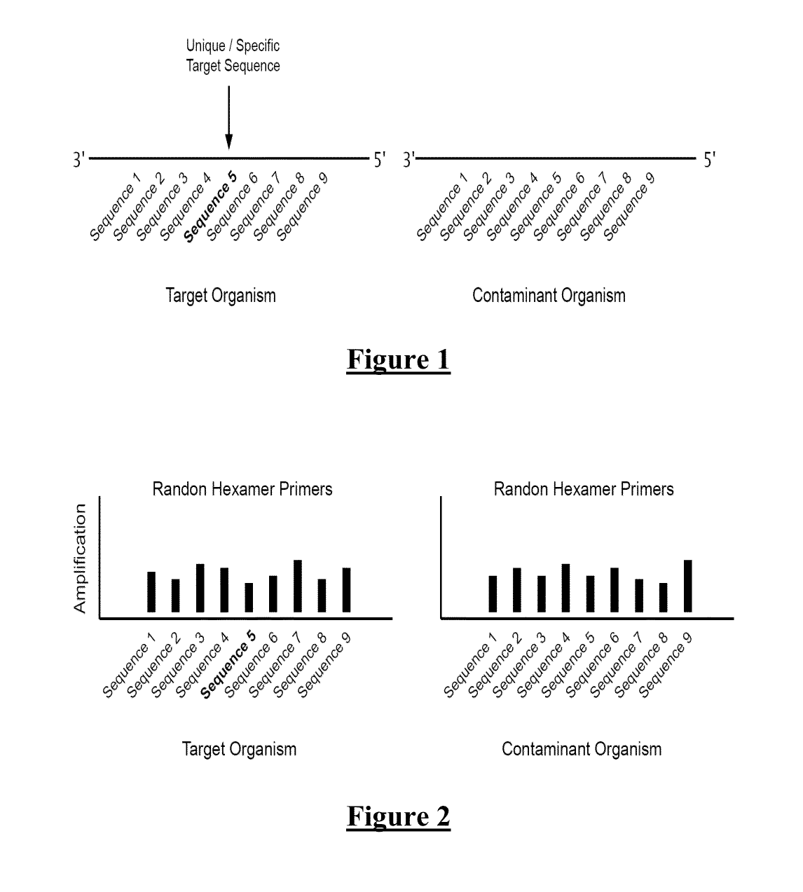

In accordance with the present disclosure, the example embodiments of FIGS. 1-3 show an annotated schematic illustration of a DNA sequence space, where a genome sequence from a target organism (see 3′-5′line target organism—left panel) and a genome sequence from a contaminant organism (3′-5′ line contaminant organism right panel) with an exemplary list of few possible sequences (indicated as Sequence 1 through Sequence 9 for each organism, in FIG. 1) to be amplified in a target and a contaminant organism is shown. In particular, as illustrated in the exemplary embodiment of FIG. 1, a typical sample can contain contaminant DNA (3′-5′ line contaminant organism right panel), target organism DNA (see 3′-5′line target organism—left panel) and a target polynucleotide in the form of a unique specific target DNA (see “Sequence 5” in FIG. 1) which is optimal for detection.

As further illustrated in the exemplary embodiment of FIG. 2, in multiple displacement amplification, sample amplification by Φ29 DNA polymerase with random hexamer primers can proceed without bias or non-representation. In the exemplary embodiment of FIG. 2, amplification comprises techniques such as Multiple Displacement Amplification (MDA), sample amplification by φ29 DNA polymerase with random hexamer primers, performed on the exemplary sample of FIG. 1. Amplification, represented as bars, shows amplification of all sequences with no one sequences favored including the unique specific target “Sequence 5”.

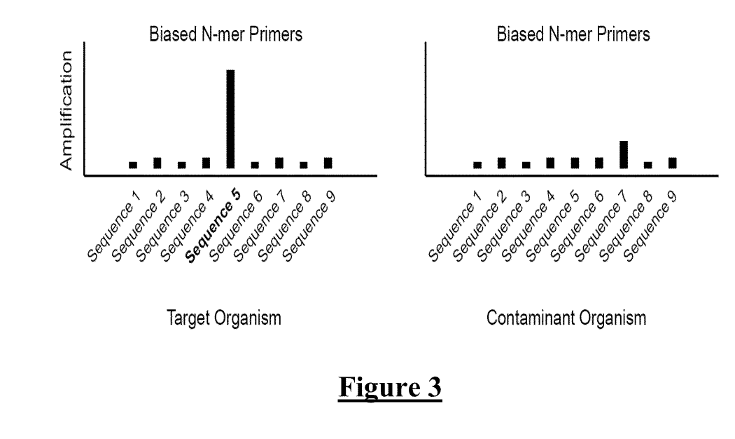

However, in biased N-mer amplification, sample amplification by Φ29 DNA polymerase with biased N-mer primers can proceed with emphasis to unique specific target DNA (see “Sequence 5” in FIGS. 1-3) that is optimal for detection, as illustrated in the exemplary embodiment of FIG. 3. As further illustrated in the exemplary embodiment of FIG. 3, some fraction of contaminant DNA (right panel), target organism DNA (left panel) and target organism DNA around the unique specific target DNA (see “Sequence 5” in FIGS. 1-3) can also be amplified. However, as shown in the illustration of FIG. 3, the biased N-mers provide a selection of polynucleotides from the sample (herein also first set of polynucleotides) wherein the presence of the unique specific target DNA is increased and the false negatives are minimized. In the exemplary embodiment of FIG. 3, amplification comprises techniques such as MDA, sample amplification by a polymerase, for example φ29 DNA polymerase, with N-mers primers obtainable with methods and systems herein described, performed on the exemplary sample of FIG. 1.

In accordance with some embodiments of the present disclosure, in order to determine the statistically a minimum oligonucleotide length yielding a unique primer sequence within the pathogenic genome, the following model can be used. The following model uses an array μ′ comprising independent and random variables with frequencies μ′=(μₐ′,μ_{c}′,μ_{g}′,μₜ′), to describe a “random” segment in the pathogen or target genome. A “random” variable can be viewed as a variable that does not have a fixed value and whose value is subject to change due to mathematical randomness. In this model, each variable in the random segment is considered “independent”, such that information pertaining to one variable in the random segment does not provide information pertaining to any other variable in the random segment. For example, in the array μ′, μ′ₐ can represent the frequency distribution of nucleotide A, μ′_{b} can represent the frequency distribution of nucleotide B, and so on. In accordance with several embodiments of the present disclosure, the array μ′=(μₐ′,μ_{c}′,μ_{g}′,μₜ′) can be used to represent the frequency distribution of the target genome, where μ′ is an array comprising the frequency distribution of different nucleotides (e.g., adenine, cytosine, guanine, and thymine). In accordance with the present disclosure, frequency distribution of N-mers can be defined as the number of times each N-mer appears in a given genomic sample. A similar model of random variables with frequencies v=(vₐ,v_{c},v_{g},vₜ) can be considered to describe a “random” segment of the contaminant genome. As well known in the art, in the context of the present disclosure, the entropy of the frequency distribution μ′ can be defined as:

$$H ( {μ}^{′} ) = - {\sum }_{i ∈ \{ a , c , g , t \}} {μ}_{i}^{′} ln {μ}_{i}^{′} . \qquad ( 1.1 )$$

In equation (1.1), the entropy of the frequency distribution can be maximized for the uniform distribution μ=(¼,¼,¼,¼). A deep result in information theory suggests that the number of different sequences of length m′ in a large genome can be given by exp[m′H(μ′)], contrary to 4^{m′} expected from the uniform distribution. If it is assumed that, there are for example, G′ bases in the pathogenic genome or the target genome, the following equation (1.2) can be satisfied based on suggestions from information theory, to make all the sequences of length m′ distinguishable within a genome. Therefore,

exp[m′H(μ′)]≧G′, (1.2)

### Consequently,

$${m}^{′} \geq \frac{ln {G}^{′}}{H ( {μ}^{′} )} . \qquad ( 1.3 )$$

In equation (1.3), since the uniform distribution can make the sequences of length m′ as random as they can be as compared to other probability distributions, the uniform frequency distribution μ=(¼,¼,¼,¼) can minimize the right hand side of equation (1.3). Therefore, if, for example, the pathogenic genome or the target genome contains M bases with a uniform distribution, by symmetry, a primer of length m′ occurs in M/4′ positions within the pathogenic genome. In such case, a 10-mer corresponds approximately to a unique position in a genome of size 4¹⁰=2²⁰≈10⁶. With a complete sequence for the pathogenic or the target genome, unique primers for the diagnostic region of interest can be determined empirically. However, when a complete sequence is not available and/or analysis of the complete sequence is impractical, the result from equation (1.3) can estimate a minimum length at which one can begin looking for unique primer sequences. One can also look for unique primer sequences at lengths higher than the minimum length. Length at which to look for unique primer sequences can depend on such considerations as computational efficiency/complexity considerations and in which application or applications the primers are to be utilized. Furthermore, denoting the total length of the contaminant genome as G, the number of primer sites in the contaminant can be:

$$n = n ( L , μ ) = G exp ( L {\sum }_{n ∈ N} {μ}_{n} ln {v}_{n} ) , \\ where , \\ L = {\sum }_{n ∈ N} L ( n ) , \qquad ( 1.4 )$$

In the above equation (1.4), it has been assumed that the count L(n) of nucleotide is nεN for oligonucleotide primer of length L=Σ_{nεN}L(n). Additionally, it has been assumed that the empirical composition of the primer is μ=(μₐ,μ_{c},μ_{g},μₜ), where μₙ=L(n)/1 L and L and μ are independent variables available to control the number of primer sites in the contaminant.

In accordance with some embodiments of present disclosure, to minimize the number of contaminant sites that match the primer, the nucleotides in the primer (for example, primer composition μ) can correspond to the most infrequent nucleotides in the contaminant genome (composition υ). For example, the human genome is about 0.42 GC. If the primer is relatively AT-rich, the number of times the primer sequence occurs in the human genome can be relatively small. As well known in the art, in order to minimize the number of contaminant sites that match the primer, the suggested steps are as follows:
(1) Some minimum “reasonable” sequence length around the diagnostic sequence can be determined to obtain a “diagnostic region” from which primers can be selected. “Reasonable” sequence length can be determined by a skilled person in the art.
(2) By using equation (1.3) a minimum primer length m′ sufficient to yield unique pathogenic primer sequences can be determined. Once the minimum primer length m′ is determined, the oligonucleotides of increasing size from the diagnostic region to the pathogenic genomes can be compared, until the comparison produces some “reasonable” number of candidate oligonucleotides, from which primers can be selected. “Reasonable” number of candidate oligonucleotides can be determined by a skilled person in the art.
(3) The primers can be selected from among the candidate oligonucleotides, by biasing their nucleotide composition away from the contaminant genome composition as far as possible. For example, for the relatively AT-rich human genome, the primers can be biased so as to contain as much GC as possible. Moreover, selection of the location of the PCR primers relative to the pathogenic genome site can be performed based on the desired amplification method and a desired amplification product as well as based on other variable related to the target amplication identifiable by a skilled person. In particular, if a short amplification product and/or fast amplification is desired, PCR primers are typically selected to be at a location that is close to the pathogenic genome site.
(4) In some applications, amplification can be performed with termination and the method further comprises mixing dideoxynucleotides and deoxynucleotides to the polymerase-target sample mixture. Dideoxynucleotides can be used as chain terminating inhibitors for DNA polymerase. If dideoxynucleotide sequencing termination is considered, the nucleotides can be contaminated with the single dideoxynucleotide base corresponding to the smallest relative composition μ/υ, so that the corresponding nucleotide is rarest in the diagnostic region relative to the contaminant genome. The size of synthesized fragments affects target amplification kinetics or products per time. To control the length distribution of synthetic fragments, a skilled person can adjust the ratio of dideoxynucleotides to deoxynucleotides. For example, a ddNTP:dNTP ratio of 100:1 to 10,000:1 can be used to synthesize fragments of about 10,000 bases or more.
(5) The relative concentration of the dideoxynucleotide base that maximizes the PCR effectiveness can be shown to be q₀=[μ(b+1)]⁻¹. In this context, b can be assumed as the reasonable sequence length plus the length of the diagnostic sequence. Thus, the overall frequency of the contaminating dideoxynucleotide base in the nucleotide mix is q₀μ=(b+1)⁻¹, making the average termination distance b+1. The reaction terminates near the end of the diagnostic region.
(6) Finally, one way to reduce the number of primer sites in the contaminant genome is to increase the length of the primers, so there is a tension between longer primers (reduced noise from the contaminant genome) and shorter primers (increased reaction speed, if shorter primers have a faster on-rate). The original branch-chain polymerization reaction is not sequence-specific, however, short primers can anneal to more sites per genome than long primers.

It is noted that, although the suggested steps above are given with reference to minimizing the number of contaminant sites, in some cases the steps can be performed to reduce but not necessarily minimize the number of contaminant sites. For example, although minimum lengths can be determined or estimated for the “reasonable” sequence length and primer length (e.g., in steps (1) and (2) above), the respective minimum lengths can serve as a starting point and values for the lengths greater than the minimum lengths can be used in the steps above. Actual values of the lengths that are utilized in the steps are generally dependent on application in which the steps are performed and can be determined by a skilled person in the art.

In some embodiments, the method of selecting N-mers for amplification comprises determining frequency distribution of N-mers in the target to obtain a target N-mer distribution; determining frequency distribution of N-mers in contaminants to obtain a contaminant N-mers distribution; and selecting a set of N-mers based on the target N-mer distribution and on the contaminant N-mers distribution.

As previously indicated, in accordance with the present disclosure, frequency distribution of N-mers can be defined as the number of times each N-mer appears in a given genomic sample. Determining the frequency distribution of N-mers involves obtaining the sequences of all DNA contained in a genomic sample. A database of N-mers can then be created, containing N length sequences made up of all possible combinations of the types of nucleotides that are present in the genomic sample. This includes the DNA nucleotides adenine, cytosine, guanine, thymine, RNA specific uracil and other DNA and RNA analogs. If the number of all possible types of nucleotides is m, then the number of possible N-mers is m^{N}. The frequency distribution of the N-mers in the genomic sample can be obtained by probing the genomic sequences, where probing the genomic sequences can be done by using each N-mer as search string. The number of matches found this way can then be counted and recorded, which in turn will represent the frequency distribution of the N-mers in the genomic sample.

In particular, to determine the frequency distribution of N-mers (with fixed length N) in a target sequence, a database can be created which contain all possible sequences of length N (N-mers) that are found within the target sequence. The frequency of occurrence of each of these N-mers can then be calculated by searching for the N-mers within the target sequence.

In accordance with the present disclosure, to determine the frequency distribution of N-mers in the contaminant DNA, first the DNA sequences of the contaminant genome can be obtained from the knowledge of the distribution of different organism's DNA in the sample. A skilled person is able to identify the most likely contaminant genomes in a sample. For example, contaminant DNA in a sample of human blood can consist of human DNA and infectious disease agents such as viruses, bacteria, and/or parasites. Consequently, a database of N-mers of a given length N can be created which includes all possible polynucleotides of length N that are present within the contaminant DNA, and the frequency of occurrence of each N-mer in the contaminant genome can then be determined by probing the contaminant genomes using the N-mer sequence as a search string.

In some embodiments identifying N-mers herein described can be performed by a method which follows the following steps: determining frequency distribution of N-mers in the target to obtain target N-mers and target N-mer distribution; determining frequency distribution of the target N-mers in contaminants to obtain a contaminant N-mers distribution specific to the target N-mers; and selecting a set of N-mers based on the target N-mer distribution and the contaminant N-mers distribution specific to the target N-mers. In such cases, to determine the frequency distribution of N-mers (with fixed length N) in a target sequence, a database containing all possible sequences of length N (N-mers) that are found within the target sequence can be created. The frequency of occurrence (or frequency distribution) of each of these N-mers in the target can then be calculated by searching for the N-mers within the target sequence. Consequently, frequency distribution of all possible N-mers (9≦N≦16) specific to the target sequence within the contaminant DNA can be determined by probing the contaminant genome using the N-mer sequence as a search string. Once the frequency distribution of N-mers in the target and the frequency distribution of all possible N-mers specific to the target sequence within the contaminant DNA is determined, groups of N-mers with biased amplification can be selected by comparing the frequency distribution of all possible N-mers of the specific one or more targets and their adjacent sequences to the frequency distribution of all possible N-mers specific to the target sequence within the contaminant DNA. Some examples of the algorithms are identifiable to a skilled person in the art including open-source and proprietary algorithms, for example those described in references [10, 12 and 13], incorporated herein by reference in their entirety.

In some embodiments, a skilled person can amplify the whole genome of the target organism. In some embodiments, a skilled person can amplify a part or fraction of the genome. In embodiments where the whole genome of the target organism is amplified, the genome can be small, for example, less than 10K bases such as viral genomes. In embodiments where the a part or fraction of the genome is amplified, the genome can be large, for example, greater than 10K bases and in such cases a specific “region of interest” can be identified within the target genome. This specific region can be chosen to represent certain genomic features that are unique to the target organism such as unique identifying sequences or specific fingerprints for the desired target. After a specific “region of interest” is identified, adjacent sequences in both sides of the “region of interest” are identified and added to the target sequence. Adjacent sequences can be included to minimize noise in the DNA amplification and increases the probability of identifying N-mers that work for all variations or quasi-species of the target. A desired length of the adjacent sequences can depend on the total number of variations or quasi-species identifiable by the skilled person. For example, the length of the adjacent sequences can be dependent on the amount of target species to target species variation. For target species with low variation (such as bacteria with low mutation and/or evolution rates), the adjacent sequence length will be shorter than for target species with high variation (such as viruses with high mutation and/or evolution rates), for example 1-2 Kb vs. an entire genome on the order of 10 Kb. A skilled person picks the range and length of the adjacent sequence in order to achieve rapid amplification and accommodate variations within quasi-species. The region of interest and the adjacent sequences on both sides comprise the target sequence.

An initial genomic collection can be obtained, for example, by downloading a complete organism or genome sequences from a database. Publicly available databases include NCBI Genbank, the Integrated Microbial Genomics (IMG) project at the Joint Genome Institute, the Comprehensive Microbial Resource (CMR) at the JC Venter Institute, The Sanger Institute in the United Kingdom and The European Bioinformatics Institute (EMBL-EBI). In-house databases containing the genome collections of interest can be used as well.

In some embodiments, biased N-mers identifiable by the method and systems herein described can be used in a method and system of amplifying a target nucleic acid.

In some embodiments, at least one pair of primers having sequences of N-mers biased for the target nucleic acid selected by a method herein described with a sample to produce a primer-target sample mixture; and incubating the primer-target sample mixture under conditions to promote hybridization between the primers and the target sequence in the primer-target sample mixture. A skilled person can select the at least one pair of primers to hybridize with a sample under the reaction conditions, such as high polymerase activity, sufficient polymerase lifetime, and diffusion limited annealing of primers to targets. For example, the skilled person can select primers with a Tₘ that in the range of the annealing temperature of the hybridization reaction. A skilled person can select primers that are not prone to dimerization, hairpin formation, or mispriming. A skilled person can also design the hybridization reaction to select a temperature to produce maximal production of target amplification products within a defined window of time.

Amplification of low quantities of DNA to detectable quantities of DNA can be for example performed by isothermal or thermocycle methods. Some example of such methods can be found in references [2] and [4] each of which is incorporated herein by reference in its entirety. Exemplary isothermal methods known in the art include Nucleic Acid Sequence-Based Amplification (NASBA), Helicase Dependent Amplification (HDA), Recombinase polymerase Amplification (RPA), Rolling Circle Amplification (RCA), Cross-Priming Amplification (CPA), Smart Amplification (Smart-AMP), Ramification Amplification (RAM), Strand Displanement Amplification (SDA), and Isothermal Chain Amplification (ICA). Exemplary thermocycle methods include polymerase chain reaction (PCR) or nested PCR. Exemplary polymerase classes used in the thermocycle methods include mesophilic and thermophilic classes. Among the various methods, those based on priming with random hexamers and amplification by φ29 DNA polymerase can be used with particular reference to diagnostic application. Such isothermal methodology is termed as “Multiple Displacement Amplification” (Refs 5-9). Exemplary amplification methods are the one described in Current Opinion which can be found in references [6] and [7], each of which is incorporated herein by reference in its entirety. Exemplary polymerases comprise φ29 DNA polymerase and/or variants that can be produced with various methods (e.g. by directed evolutionary methods and additional methods identifiable by a skilled person). In some embodiments, a DNA polymerase used can have high processivity and generates DNA fragments over 10 kilobases in length with random hexamer priming. However, any isothermal amplification method that uses oligonucleotide primers to direct and initiate DNA synthesis can be utilized in methods and systems of the disclosure. Some example of such methods can be found in reference [2], incorporated herein by reference in its entirety.

In particular the biased amplification can be performed by mixing a polymerase with the primer-target sample mixture to produce a polymerase-target sample mixture, and incubating the polymerase-target sample mixture that promote replication of the target sequence. In particular, in some embodiments the polymerase is an isothermal polymerase.

In some embodiments, amplification performed with biased N-mers of the disclosure allows obtaining a selection of a first set of polynucleotides from the polynucleotides of the samples in which false negatives for the target polynucleotide is minimized.

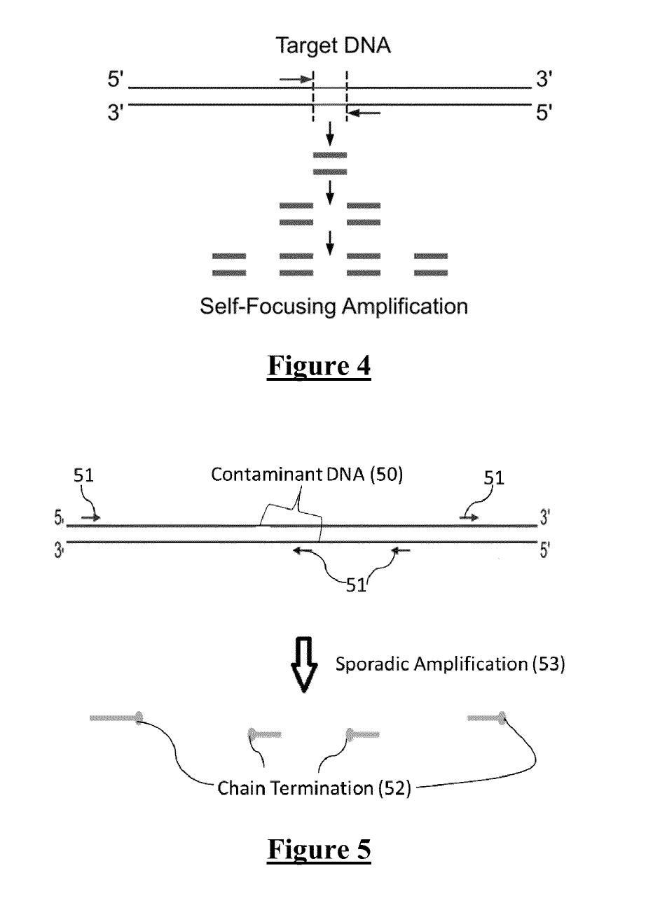

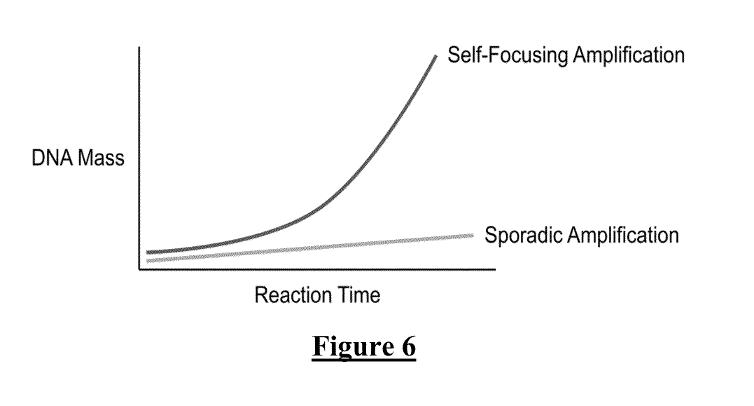

Reference is made to the illustration of FIGS. 4 to 6 to illustrate an exemplary biased N-mer amplification which makes use of one or more primer sets that bind to complimentary strands of DNA and polymerases that replicate desired segments of DNA with bias to species of interest which is in particular provided by the target polynucleotide, and contaminant. In the illustration of FIG. 4, a schematic illustration is provided of the mechanism of self-focusing amplification cascade. In particular, with prior knowledge of both genomes, semi-specific oligonucleotide primers (arrows in 5′ to ′3 direction and 3′ to 5′ direction) that consist of N-mers (with 9≦N≦16) can be selected to amplify unique target sequences of interest over contaminating DNA (FIG. 2). When such semi-specific primers bind to complimentary target DNA in close proximity (dotted lines), a self-focusing amplification cascade (thick black lines) takes place. FIG. 5 shows a schematic representation of an exemplary amplification of contaminant polynucleotide (50) performed with probes or primers (51) having sequences from biased N-mers herein described. However, as illustrated in the schematic of FIG. 5, when such primers (51) bind to complimentary contaminant DNA (50) in a scattered pattern, only sporadic amplification (53) of contaminant DNA (53) takes place. In particular, the schematic illustration of FIG. 5 shows the semi-specific, self-focusing isothermal amplification of DNA with the primers (51) of FIG. 4 performed on a contaminant DNA (50) present in the same sample of the DNA. Sporadic amplification of contaminant DNA will occur for N-mers that are separated by distances and that are greater than the average length of single-stranded amplicons. The ratio of dideoxynucleotides to deoxynucleotides (ddNTPs/NTPs) determines the statistics of chain termination (52) as denoted by a solid circle. Lower ratios result in longer synthetic fragments. Higher ratios result in shorter synthetic fragments. For self-focusing to take place, the distance between opposing primers are typically no greater than the average length of single-stranded amplicons. The results of the self-focusing amplification performed with biased N-mers herein described according to the approach illustrated in the schematic of FIG. 4 and FIG. 5 is further illustrated in FIG. 6 wherein the self-focusing amplification of the target polynucleotide of FIG. 4 is plotted versus the sporadic amplification of contaminant polynucleotide of FIG. 5. In particular, the schematic illustration of FIG. 6 shows the semi-specific, self-focusing isothermal amplification of DNA with the primers of FIG. 4 performed on a contaminant DNA present in the same sample of the DNA. In the illustration of figure FIG. 6 for self-focusing to take place, the distance between opposing primers are typically no greater than the average length of single-stranded amplicons.

In some embodiments, the amplification can be performed with termination and the method further comprises mixing dideoxynucleotides and deoxynucleotides to the polymerase-target sample mixture.

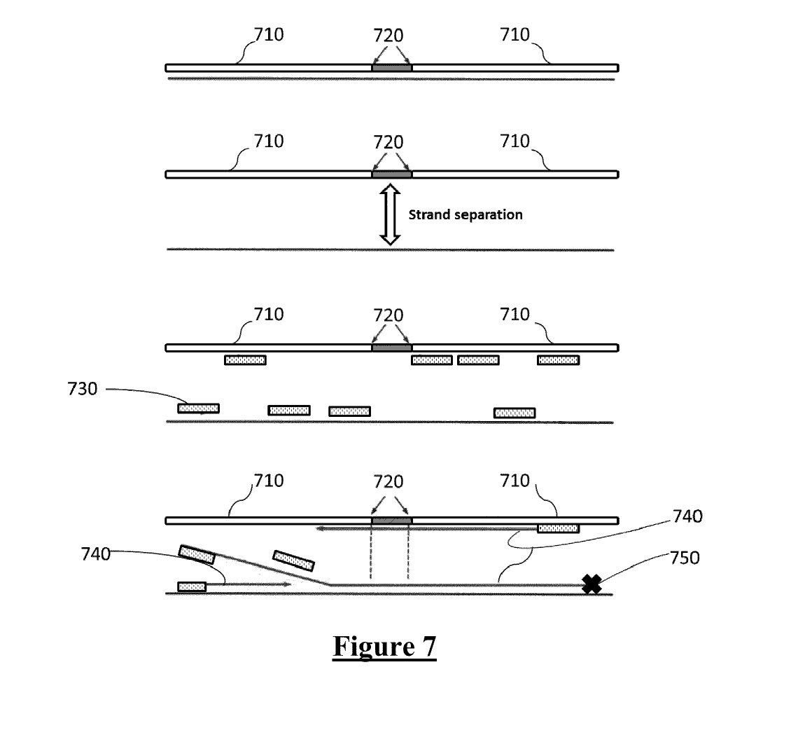

A schematic illustration of exemplary embodiments, in which biased amplification performed with chain termination to suppress the contaminate DNA is also provided in FIGS. 4 and 5 and in FIG. 7.

In particular, FIG. 7 shows a schematic description of an exemplary biased N-mer amplification of DNA with termination of a unique specific target DNA (710) and its adjacent DNA (720) according to embodiments herein described. DNA synthesis is primed by biased N-mers (primers, 730). Exponential amplification, denoted by the extending arrow (740) occurs by a “branching” mechanism. φ29 DNA polymerase acts to extend primers (N-mers, 730), as it displaces downstream DNA products. The ratio of dideoxynucleotides to deoxynucleotides (ddNTPs/NTPs) determines the statistics of chain termination as denoted by an “X” symbol (750). Lower ratios result in longer synthetic fragments. Higher ratios result in shorter synthetic fragments.

In particular, an isothermal amplification of DNA entails primer annealing, polymerases attachment, strand displacement/elongation and polymerase detachment. When target sequences of interest are relatively short (720), polymerase cycling can be increased by adding dideoxynucleotides (ddNTP) in a small ratio compared to deoxynucleotides (dNTP). In particular, to control the length of synthetic fragments, the method of the disclosure incorporates four deoxynucleotide bases (ATP, TTP, GTP, CTP) and a low concentration of a chain terminating or dideoxynucleotide bases (ddATP, ddTTP, ddGTP, ddCTP). As in Sanger DNA sequencing, the relative concentration dideoxynucleotide (ddNTP) to deoxynucleotide (NTP) bases determines the statistics of chain termination, some example of which can be found in reference [11], incorporated herein as reference in its entirety. In particular, lower ddNTP/NTP ratios can result in longer synthetic fragments. However, higher ddNTP/NTP ratios can result in shorter synthetic fragments (FIG. 7). In particular in some embodiments, a ratio of dideoxynucleotides to deoxynucleotides associated to a desired length of the synthetic fragments to be amplified can be calculated (calculated ratio) using for example computer programs identifiable by a skilled person. The calculated ratio can then be used to determine the proporaration of dideoxynucleotides to deoxynucleotides that are used in the amplification reaction. For example, a ddNTP:dNTP ratio of 100:1 to 10,000:1 can be used to synthesize fragments of about 10,000 bases or more.

Such chain termination (750) in the illustration of FIG. 7 and light gray circles at one end of light gray lines of FIG. 5) can reduce the overall length of sporadic amplification products from contaminant DNA and thereby enhance the self-focusing amplification cascade (thick black lines of FIG. 4).

In embodiments herein described, amplification performed with biased N-mers herein described can be used in a method to detect at least one target nucleic acid in a sample. In particular, in embodiments of the method to select at least one target nucleic acid in which use of the N-mers of the disclosure results in a selection of a first set of polynucleotide that is biased towards the at least one nucleic acid of choice.

In particular, providing a set of polynucleotides biased towards the at least one target nucleic acid of choice can be obtained by performing polynucleotide amplification on the sample with at least one pair of primers having sequences of N-mers biased for the at least one target selected with the method herein described to provide a first set of polynucleotide. In some embodiments, the amplification is performed with N-mers biased towards target polynucleotides that are flanked by adjacent sequence. An exemplary schematic illustration of those embodiments is provided in FIG. 7.

According to several embodiments of the present disclosure, statistical quantities, for example, true positives (TP), false positives (FP), true negatives (TN), false negatives (FN) can describe the performance of a statistical test of binary classification. For example, true positives can be the number of cases that are correctly identified as positives. However, false positives can denote the number of cases that are identified as positives, but are in fact negatives.

In accordance with the present disclosure, during the amplification of the target, false negatives can arise if certain N-mers which are present in the target DNA with higher frequency of occurrence have been omitted from the primer pool. In such cases, the omitted N-mers can also have higher frequency of occurrence in the contaminant DNA as well. Therefore, in such cases, the degree of bias corresponding to the N-mer selection can initially be reduced to include all N-mers present in the target sequence that have equal or higher frequency in the target DNA compared to the contaminant DNA. Consequently, the frequently occurring oligonucleotides in the target sequence can be amplified. In such amplification process, the contaminant DNA can be partially amplified as well. Although, in such cases, false positives will increase due to partial amplification of the contaminant DNA, false negatives will be minimized due to the reduced degree of bias involved in selecting the N-mers.

In accordance with the present disclosure, the amplified gene can then be selectively amplified with respect to the target sequence by using primers, which are heavily biased towards the target sequence. This process can include the N-mers which are significantly more frequent in the target sequence compared to the contaminant sequence. In some embodiments, this step will reduce the number of false positives and therefore the amount of unwanted amplification of contaminant DNA while at the same time ensure an efficient amplification of the target DNA.

The reduction of the false positive can ensure the proper amplification of all desired parts of the target genome while suppressing the amplification of spurious contaminant genome. Therefore, a “low bias” N-mer selection can be slowly increased in several successive amplification steps to improve the true positives while keeping the false negatives to a minimum at the same time. In some embodiments, the amplification in each step can be controlled (kept at a lower level compared to the one step process) to reach an optimum balance between false positives, and true positives in each successive step.

In some embodiments, a target nucleic acid can be detected from a first set of polynucleotides selected from the sample to minimize the false negative by contacting the first set of polynucleotide with one or more probe specific for target polynucleotide. In some embodiments, in which detection of more than one target polynucleotide is desired, one or more probes specific for each target polynucleotide to be detected can be contacted with the first set of polynucleotides, to detect the desired target polynucleotides.

In this context, as known in the art, mathematically, sensitivity and specificity are parameters for determining the performance efficiency of a statistical test of binary classification. Sensitivity, also called the true positive rate, can be defined as the proportion of true positives among the total number of identified positives. Specificity, known as the true negative rate, can be defined as the proportion of true negatives among the total number of identified negatives. For example, in a blood test to identify a specific disease condition, sensitivity can be the proportion of patients having the disease among the total number of patients diagnosed positive by the blood test. Likewise, sensitivity is given by the proportion of healthy individuals among the total number of people diagnosed negative by the blood test.

In the present disclosure, the terms “specific”, “specifically” or “specificity” is also with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that can be present. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of. The term “specific” as used herein with reference to a sequence of a polynucleotide refers to the unique association of the sequence with a single polynucleotide which is the complementary sequence. By “stable complex” is meant a complex that is detectable and does not require any arbitrary level of stability, although greater stability is generally preferred.

The terms “detect” or “detection” as used herein indicates the determination of the existence, presence or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “signal” as used herein indicates the signal emitted from the label that allows detection of the label, including but not limited to radioactivity, fluorescence, chemiluminescence, production of a compound in outcome of an enzymatic reaction and the like. The terms “label” and “labeled molecule” as used herein as a component of a complex or molecule referring to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image.

The term “detection limit” as used herein indicates the minimal number of targets in the sample that will result in positive detection.

Exemplary methods of detection include but are not limited to optimal methods such as Fluorescence Resonant Energy Transfer (FRET), fluorescent imaging and microscopy, nanoparticle resonance, surface plasmon resonance, and quantum dot, enzymatic methods, electromagnetic methods such as Nuclear Magnetic Resonance (NMR), and electrical detection methods utilizing resistance in an array based, electrochemical, nanowire, or graphene apparatus.

In some embodiments, methods and systems herein provided can be performed to detect at least one target polynucleotide. In some embodiments, the methods and systems herein described can be performed to detect a plurality of targets in a multiplexed approach. The selection of a plurality of primer pairs is similar to the selection of one primer pair. The iterative process is performed by serial (one pair per cycle) and/or parallel (multiple pairs per cycle) selection processes. In the process of selection of biased N-mers, each sequence in a multiplex scheme provides independent information.

In some embodiments, the detected target polynucleotide is a biomolecule and in particular a biomarker associated to an organism and in particular a microorganism or particle. In some embodiments, primers are selected to detect a target group of polynucleotides associated with a grouping of organisms (and in particular microorganisms) or particles. In particular, the term “target group” as used herein refers to a group of polynucleotide specifically associated to one or more organisms or viral particles with related sequences. By way of example and not of limitation, a target group can be a viral family or a bacterial family or the group of related polynucleotides. In particular, a target family comprises the family classification according to the NCBI (National Center for Biotechnology Information) taxonomy tree. A target group can also comprise a viral, bacterial, fungal, or protozoal sequence group classified under a taxonomic node other than family.

In some embodiments, a target polynucleotide can be converted from RNA into DNA e.g. by reverse transcription, before proceeding with the N-mers identification. In some of those embodiments N-mers and related probes and amplification and detection methods herein described can be used to perform a semi-specific paired N-mer primers with about 9≦N≦16 to amplify a specific region of target DNA or RNA that was converted to DNA by prior reverse transcription. In some embodiments, N-mers and related probes and amplification and detection methods herein described can bind to RNA and be used in combination with reverse transcriptase.

In accordance with the present disclosure, a mixture of random primers, for example random hexamers, can be used in combination and/or mixture with biased N-mers. In particular, in some embodiments, random hexamers can be added to the reaction mixture together with biased N-mers primers, suitable reagents, and a polymerase. In some embodiments, random hexamers can be added to the reaction mixture together with suitable reagents and polymerase before addition of biased N-mers primers. In these embodiments, the random primers initiate an amplification cascade and/or produce single stranded DNA and following the intiation or production of single stranded DNA, N-mers propagate biased amplification from the product of the random primers. A skilled person can calculate and adjusts ratios and/or concentrations of random primers to biased N-mer in order favor biased amplification according to the experimental design based on parameters identifiable by a skilled person.

In some embodiments, N-mers and related probes and amplification and detection methods herein described can be used to perform selection of paired semi-specific paired primers that flank the desired target DNA sequences.

In some embodiments, N-mers and related probes and amplification and detection methods herein described can be used to perform suppression of the amplification of contaminant DNA.

As disclosed herein, the biased N-mers, and related oligonucleotides and probes herein described can be provided as a part of systems to perform any assay, including any of the assays described herein directed to the detection of polynucleotides. The systems can be provided in the form of or kits of parts.

In a kit of parts, the biased N-mers, and related oligonucleotides and probes herein described and other reagents to perform the assay can be comprised in the kit independently. The biased N-mers, and related oligonucleotides and probes herein described can be included in one or more compositions, and each the biased N-mer, and related oligonucleotide and probe herein described can be in a composition together with a suitable vehicle.

Additional components can include labeled molecules and in particular, labeled polynucleotides, labeled proteins and enzymes, labels, microfluidic chip, reference standards, and additional components identifiable by a skilled person upon reading of the present disclosure.

In some embodiments, detection of a target polynucleotide can be carried either via fluorescent based readouts, in which the captured polynucleotide is labeled with fluorophore, which includes, but not exhaustively, small molecular dyes, protein chromophores, quantum dots, gold nanoparticles, and paramagnetic nanoparticles. Additional techniques are identifiable by a skilled person upon reading of the present disclosure and will not be further discussed in detail.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

In some embodiments, biased N-mers herein described and related methods and systems can be used within a device for point of care diagnostics. In some embodiments, the device is used in in vitro and in situ bioassays. An exemplary device comprises a interface to detect the at least one target polynucleotide in a sample input containing the sample input and reagents necessary for amplification, detection, and adjusting excess absorption. The system comprises a disposable stick that houses an interface to detect the at least one target polynucleotide, a reusable stick reader or dock, and an interface to collect data from the stick to a computer or smartdevice. In some embodiments, the computer and smartdevice has a program or application to process the data collected from the stick. In some embodiments, the computer or smart device syncs the data to a cloud-based computing. In some embodiments the device can be the devices described and/or claimed in U.S. Provisional Application No. 61/668,904 filed on Jul. 6, 2012 incorporated by reference in its entirety.

The term “smart device” as used herein indicates an electronic device configured to have internal memory, communication capabilities, and computing capabilities using artificial intelligence. Some examples of smart devices which can be used in the context of the present disclosure can be iPhone®, iPad®, Android® phone etc.

The term “cloud-based computing” as used herein indicates an internet based computing method in which information is collected from the internet through web-based tools and applications, rather than a direct connection to a server. An example of such server is provided by Amazon Web Services.

In some embodiments, the N-mers, and related probes, methods and systems herein provided are used when rapid amplification and detection of one or more target polynucleotides is desired. In other embodiments, N-mers, and related probes, methods and systems herein provided when economy of reagents related to detection of one or more target polynucleotides is desired.

In some embodiments, the N-mers, and related probes, methods and systems herein provided are comprised in a non-portable or portable device. In some embodiments, the non-portable or portable device is a point-of-care or laboratory device.

In some embodiments biased N-mers based probes are used for point-of-care molecular diagnostics, it is not necessary nor desirable to amplify all DNA contained within samples. In those embodiments it is desireble to amplify specific and relatively short DNA sequences within samples. One way to achieve this goal is to use relatively long specific primers (usually 18-30 nucleotides) at high concentrations in combination with φ29 DNA polymerase (or other suitable polymerase). Yet relatively long specific primers (even at high concentrations) can reduce DNA amplification rates, because longer primers have higher melting temperatures, Tm=4(G+C)+2(A+T) C°, and/or slower annealing efficiencies to complimentary DNA (Ref 10).

Another way to achieve the desired goal is to use intermediate length primers in combination with a polymerase, for example φ29 DNA polymerase. Yet intermediate length primers lack overall specificity and the number of all possible sequences scales as 4^{N}, where N is nucleotide length, which makes the synthesis and supply of all primer sequences less than practical. For example, random decamers (N=10) have 1,048,576 possible combinations, which in certain embodiments is not desired. In some of those embodiments, the use of random decamer primers does not offer a desired efficiency over random hexamer primers in combination with a φ29 DNA polymerase (or other suitable polymerase) (Ref 10). However, the selection and use of biased mixtures of intermediate length primers can offer significant benefits by achieving relatively fast annealing efficiencies to complimentary DNA and, therefore, relatively fast amplification of relatively specific DNA within samples.

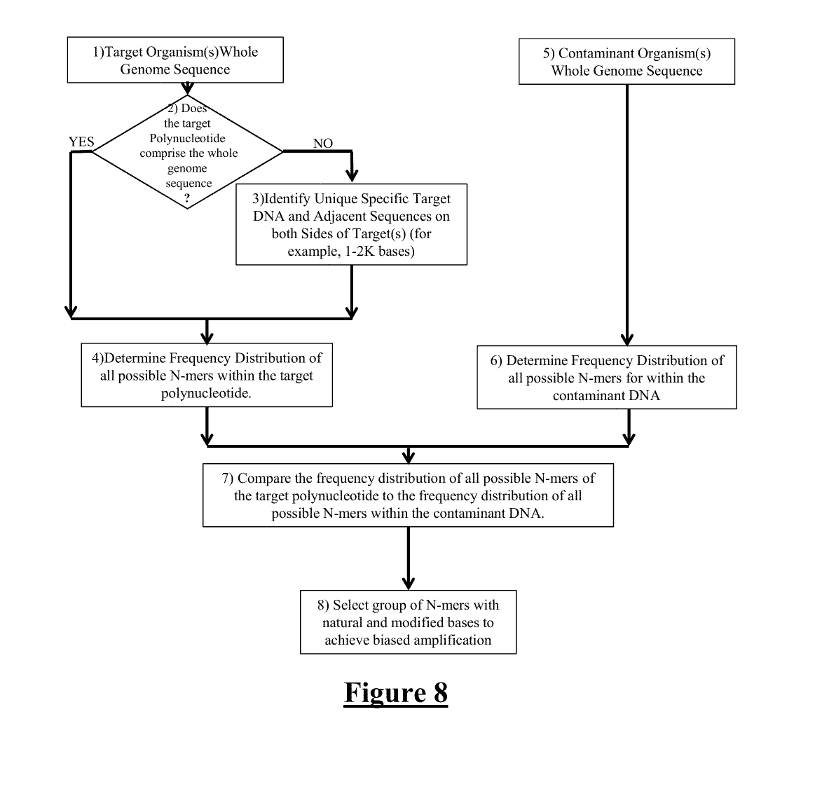

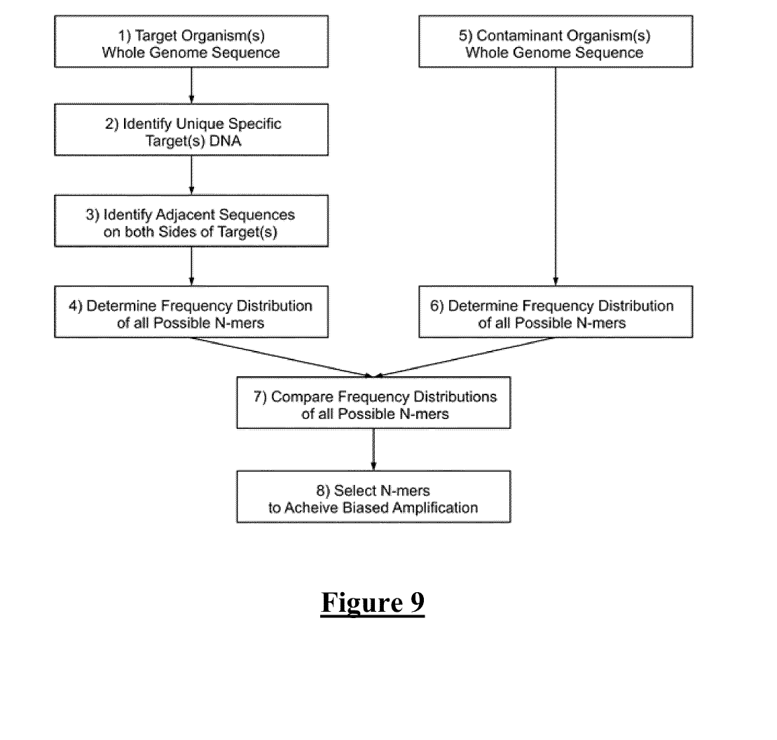

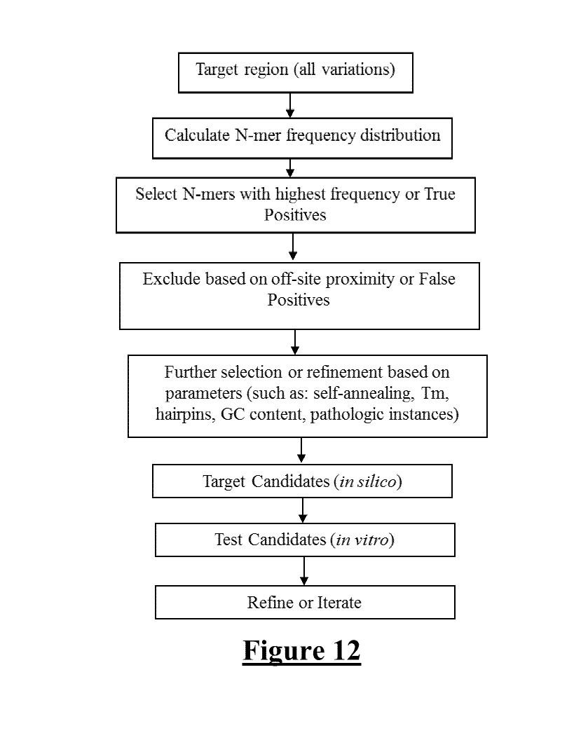

According to an exemplary embodiment of the present disclosure, FIG. 8 shows a block diagram of an exemplary method to perform biased amplification according to embodiments herein described. In the example embodiment of FIG. 8, the biased N-mers of the disclosure are used for point of care diagnostics, selection of biased N-mers can be performed with the following approach:
1) Ascertain and obtain one or more whole genome sequence of one or more target organisms of choice, (possibly including one or more plasmids and/or one or more microRNAs). If the organism and/or sequence is RNA-based, the sequence can be represented as the corresponding DNA sequence.
2) Within the one or more target organisms, identify a target polynucleotide. In some cases, the whole genome sequence of the target organism(s) can be the target polynucleotide. In other cases, a one or more specific target DNA sequence which is unique or specific for the one or more target organism and its adjacent 3′ and 5′ sequences (for example, within 1-2 kilobases) of the one or more specific target DNA sequence is the target polynucleotide. (See, for example, FIG. 9)
3) Determine the frequency distribution of all possible N-mers (for example, 9≦N≦16) within the target polynucleotide. Frequency distribution calculations which can be used in this step can be found in the references such as [10], [12] and [13], incorporated herein by reference in their entirety.
4) Ascertain and obtain at least one whole genome sequence of the contaminant DNA within samples. For example, contaminant can comprise of eukaryotic, prokaryotic, plant, viral and/or fungal DNA.
5) Determine the frequency distribution of all possible N-mers (for example, 9≦N≦16) within the contaminant DNA. Contaminant DNA can be from one or more organisms. If contaminant DNA is from more than one organism, all contaminants are considered independently. Each contaminant is considered one by one and ranked in order of probability of being present. Frequency distribution calculations which can be used in this step can be found in the references such as [10], [12] and [13], incorporated herein by reference in their entirety.
6) Compare the frequency distribution of all possible N-mers (for example, 9≦N≦16) between the unique specific target(s) and their adjacent sequence(s) from Step 4 to the frequency distribution of all possible N-mers (for example, 9≦N≦16) within the contaminant DNA from Step 6. Parameters for comparison can include N-mer length, total number of hits for each unique N-mer, and the proximity of selected N-mer pairs.
7) Select a group of N-mers (oligos) to achieve biased amplification. Selecting N-mers can in particular be performed based on one or more parameters which include location of the N-mers in target genome and one or more contaminant genomes and possibly additional parameters which are determined based on desired experimental conditions for downstream applications. In particular, selection of a group N-mers can be performed by determining the location of N-mers in target genome and one or more contaminant genomes. Location of N-mers can be, for example, annotated by the position of N-mers relative to the start of the closest open reading frame. N-mers with a ratio of high frequency of hits in the target polynucleotide (true positive) and a low frequency hits outside of the target polynucleotide (false positive) can then be selected. In cases where the target polynucleotide is the whole target genome, hits outside of the target polynucleotide include all contaminant genomes. In cases where the target polynucleotide is a specific target DNA sequence, hits outside of the target polynucleotide include all contaminant genomes and the target genome excluding the target polynucleotide. The threshold of the ratio of high frequency hits to low frequency can be determined by a skilled person based on downstream techniques. For example, a skilled person can choose N-mers to have a several fold difference between hits in the target polynucleotide and outside the target polynucleotide in order to visualize an amplification product clearly on an agarose gel.
Selection can include further parameters determined by a skilled person. For example, N-mers with a desired Tm temperature for annealing in a downstream amplification reaction can be favored over N-mers with a Tm outside a desired range of annealing. N-mers with a high likelihood of self-annealing or hairpin formation can be excluded (See FIG. 12). In addition, selection excluding parameters of N-mers for biased amplification can comprise to N-mer sequence overlap and/or close proximity of N-mer pairs.
Additional steps to select N-mers, or target candidates, can include synthesizing, polymerizing, and/or complementary nucleotides to have chosen N-mer sequences to provide primers and performing an amplification reaction with the primers and a sample containing nucleic acid from the target organism. The primers can be synthesized or polymerized and/or be composed of natural and modified bases, such as phosphorothioated bases that resist nuclease degradation. Amplification reactions can be detected for positive amplification through techniques known in the art, for example gel electrophoresis of amplification samples, sequencing, and/or RT-PCR based assays. Reactions where no amplification is detected can be retested for amplification and/or the skilled person can exclude the corresponding N-mers from the set of selected N-mers, and further selection and/or refinement can be reiterated (See FIG. 12).

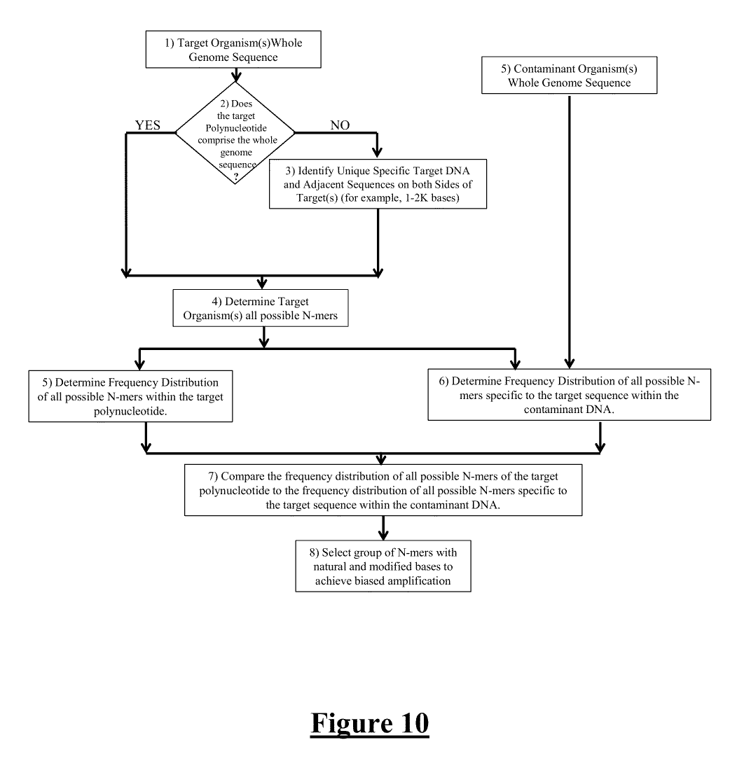

According to an examplary embodiment of the present disclosure, FIG. 10 shows an alternate embodiment of the method described in FIG. 8 to perform biased amplification. In the algorithm of FIG. 10, all possible N-mers of the target polynucleotide is calculated after following the first three steps of the previous algorithm (FIG. 9). Once the all possible N-mers of the target polynucleotide have been calculated in step 4 of this algorithm. Step 5 of this algorithm calculates the contaminant genomes whole genome sequence. In following steps 6 and 7, the frequency distribution of all possible N-mers within the target polynucleotide and the frequency distribution of all possible N-mers specific to the target sequence within the contaminant DNA will be calculated. In the next step (step 8) of this algorithm, the frequency distribution of all possible N-mers of the of the target polynucleotide will be compared to the frequency distribution of all possible N-mers specific to the target sequence within the contaminant DNA to determine the group of N-mers for biased amplification.

Functional elements of a fully integrated point-of care platform can include in some embodiments: 1) single-use test sticks and multi-use test docks for testing with objective readouts; 2) mobile device(s) and app(s) for data and/or metadata capture; 3) telcom infrastructure for two-way communications; 4) and informatics and/or cloud-base resources for user support. In addition, as previously described, the functional elements of fully integrated single-use test stick based on wicking monolith membranes include: 1) sample collection and cleanup; 2) reverse transcription of RNA to DNA and/or amplification of DNA; 3) detection, quantification and/or readout of amplified DNA; and 4) absorption of excess fluid. For an exemplary diagnostic platform, reverse transcription of RNA to DNA is used to convert RNA genomes or specific targets (from RNA viruses or microRNAs, for example) to DNA. For purely DNA-based genomes or specific targets, this step is not necessary.

Further characteristics of the present disclosure will become more apparent hereinafter from the following detailed disclosure by way or illustration only with reference to an experimental section.

In an embodiment, steps in the method of the present disclosure can be written in a variety of computer programming and scripting languages. In particular, the sequences of the N-mers and the executable steps according to the methods and algorithms of the disclosure can be stored on a physical medium, a computer, or on a computer readable medium. All the software programs were developed, tested and installed on desktop PCs and multi-node clusters with Intel processors running the Linux operating system. The various steps can be performed in multiple-processor mode or single-processor mode. All programs can also be able to run with minimal modification on most PCs and clusters. The steps outlined in FIG. 8 can be written as modules configured to perform the task. Additional steps to further optimize the method of the present disclosure can be written as additional modules to be performed in sequence or concurrently with other modules of the method.

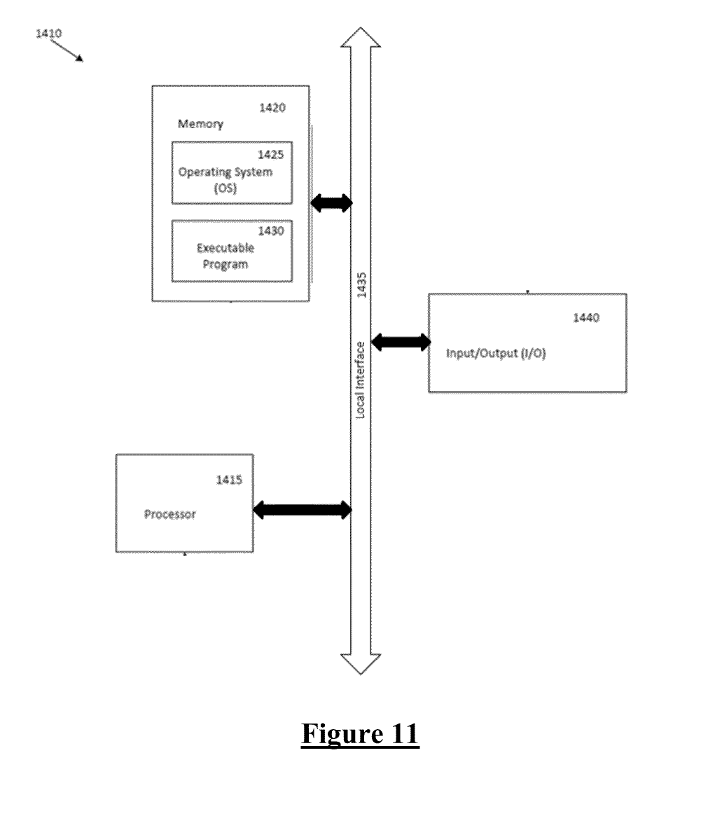

FIG. 11 shows a computer system 1410 that may be used to implement the method of the present disclosure. Certain elements can be additionally incorporated into computer system 1410 and that the figure only shows certain basic elements (illustrated in the form of functional blocks) as will be understood by a skilled person. These functional blocks include a processor 1415, memory 1420, and one or more input and/or output (I/O) devices 1440 (or peripherals) that are communicatively coupled via a local interface 1435. The local interface 1435 can be, for example, metal tracks on a printed circuit board, or any other forms of wired, wireless, and/or optical connection media. Furthermore, the local interface 1435 is a symbolic representation of several elements such as controllers, buffers (caches), drivers, repeaters, and receivers that are generally directed at providing address, control, and/or data connections between multiple elements.

The processor 1415 is a hardware device for executing software, more particularly, software stored in memory 1420. The processor 1415 can be any commercially available processor or a custom-built device. Examples of suitable commercially available microprocessors include processors manufactured by companies such as Intel, AMD, and Motorola.

The memory 1420 can include any type of one or more volatile memory elements (e.g., random access memory (RAM, such as DRAM, SRAM, SDRAM, etc.)) and nonvolatile memory elements (e.g., ROM, hard drive, tape, CDROM, etc.). The memory elements may incorporate electronic, magnetic, optical, and/or other types of storage technology. It must be understood that the memory 1420 can be implemented as a single device or as a number of devices arranged in a distributed structure, wherein various memory components are situated remote from one another, but each accessible, directly or indirectly, by the processor 1415.

The software in memory 1420 may include one or more separate programs, each of which comprises an ordered listing of executable instructions for implementing logical functions. In the example of FIG. 11, the software in the memory 1420 includes an executable program 1430 that can be executed perform the method of the present disclosure. Memory 1420 further includes a suitable operating system (OS) 1425. The OS 1425 can be an operating system that is used in various types of commercially-available devices such as, for example, a personal computer running a Windows® OS, an Apple® product running an Apple-related OS, or an Android® OS running in a smart phone. The operating system 1425 essentially controls the execution of executable program 1430 and also the execution of other computer programs, such as those providing scheduling, input-output control, file and data management, memory management, and communication control and related services.

Executable program 1430 is a source program, executable program (object code), script, or any other entity comprising a set of instructions to be executed in order to perform a functionality. When a source program, then the program may be translated via a compiler, assembler, interpreter, or the like, and may or may not also be included within the memory 1420, so as to operate properly in connection with the OS 1425.

The I/O devices 1440 may include input devices, for example but not limited to, a keyboard, mouse, scanner, microphone, etc. Furthermore, the I/O devices 1440 may also include output devices, for example but not limited to, a printer and/or a display. Finally, the I/O devices 1440 may further include devices that communicate both inputs and outputs, for instance but not limited to, a modulator/demodulator (modem; for accessing another device, system, or network), a radio frequency (RF) or other transceiver, a telephonic interface, a bridge, a router, etc.

If the computer system 1410 is a PC, workstation, smartdevice, or the like, the software in the memory 1420 may further include a basic input output system (BIOS) (omitted for simplicity). The BIOS is a set of essential software routines that initialize and test hardware at startup, start the OS 1425, and support the transfer of data among the hardware devices. The BIOS is stored in ROM so that the BIOS can be executed when the computer system 1410 is activated.

When the computer system 1410 is in operation, the processor 1415 is configured to execute software stored within the memory 1420, to communicate data to and from the memory 1420, and to generally control operations of the computer system 1410 pursuant to the software. Method of the present disclosureing and the OS 1425 are read by the processor 1415, perhaps buffered within the processor 1415, and then executed.

When the audio data spread spectrum embedding and detection system is implemented in software, as is shown in FIG. 11, the computer-executable steps of the method of the present disclosure can be stored on any computer readable storage medium for use by, or in connection with, any computer related system or method. In the context of this document, a computer readable storage medium is an electronic, magnetic, optical, or other physical device or means that can contain or store a computer program for use by, or in connection with, a computer related system or method.

Several steps of the method according to the present disclosure can be embodied in any computer-readable storage medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable storage medium” can be any means that can store, communicate, propagate, or transport the program for use by or in connection with the instruction execution system, apparatus, or device. The computer readable storage medium can be, for example but not limited to, an electronic, magnetic, optical, electromagnetic, infrared, or semiconductor system, apparatus, or device. More specific examples (a non-exhaustive list) of the computer-readable storage medium can include the following: a portable computer diskette, a random access memory (RAM), a read-only memory (ROM), an erasable programmable read-only memory (EPROM, EEPROM, or Flash memory) an optical disk such as a DVD or a CD.

In an alternative embodiment, where some or all of the steps of a method of the present disclosure to the present disclosure are implemented in hardware, the audio data spread spectrum embedding and detection system can implemented with any one, or a combination, of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

The examples set forth above are provided to give those of ordinary skill in the art a complete disclosure and description of how to make and use the embodiments of the biased n-mers identification methods, probes and systems for target amplification and detection of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### REFERENCES

1. Yager P, Domingo G J, Gerdes J. Point-of-Care Diagnostics for Global Health. Annual Reviews of Biomedical Engineering 10; 107-144:2008.
2. Niemz, A et al. Point-of-care nucleic acid testing for infectious diseases. Trends in Biotechnology 29; 240-250:2011.
3. Kim J, Easley C J. Isothermal DNA amplification in bioanalysis: strategies and applications. Bioanalysis 3; 227-239:2011.
4. Gill P, Ghaemi A. Nucleic Acid Isothermal Amplification Technologies—A Review. Nucleosides, Nucleotides, and Nucleic Acids, 27; 224-243:2008.
5. Web page: en.wikipedia.org/wiki/Multiple_displacement_amplification
6. Lizardi, P M. Multiple Displacement Amplification. U.S. Pat. No. 6,124,120: Sep. 26, 2000.
7. Hawkins T L, et al. Whole genome amplification applications and advances. Current Opinion in Biotechnology 13; 65-67:2002.
8. Dean, F B. Multiple Displacement Amplification. U.S. Pat. No. 6,977,148: Dec. 20, 2005.
9. Hutchinson C A, et al. Cell-free cloning using 29 DNA polymerase. Proc Natl Acad Sci USA 102; 17332-17336:2005.
10. Louw, T M et al. Experimental validation of a fundamental model for PCR efficiency. Chemical Engineering Science 66; 1783-1789:2011.
11. Web page: en.wikipedia.org/wiki/DNA_sequencing
12. Rosen G, Garbarine E, Caseiro D, Polikar R, Sokhansaj B. Metagenome Fragment Classification Using N-Mer Frequency Profiles, Advances in Bioinformatics 2008; 1-12: 2008.
13. Hysom D A, Naraghi-Arani P, Elsheikh M, Carrillo A C, Williams P L, Gardner S N, Skip the Alignment: Degenerate, Multiplex Primer and Probe Design Using K-mer Matching Instead of Alignments, PLoS ONE 7; 1-12:2012.

1. A method to select N-mers of about 9 to 16 nucleotides for manufacturing oligonucleotide probes biased towards a target polynucleotide of a target organism, the method comprising:
determining frequency distribution and location of N-mers in the target polynucleotide to obtain a target N-mer distribution;
determining frequency distribution of N-mers in contaminants to obtain a contaminant N-mers distribution; and
selecting a set of N-mers based on the target N-mer distribution and location, and on the contaminant N-mers distribution and location to provide a selected N-mers for the manufacturing of the oligonucleotide probes.

2. The method of claim 1, wherein the target polynucleotide comprises a whole genome sequence of the target organism.

3. The method of claim 1, wherein the target polynucleotide comprises a unique polynucleotide and adjacent sequences on the 3′ and 5′ side of the unique sequence.

4. The method of claim 3, wherein the length of the adjacent sequences is about 1-2 Kilobases.

5. The method of claim 1, wherein determining frequency distribution of N-mers in contaminants is performed by:
obtaining the whole genome sequence of at least one contaminant organism or part of a whole genome sequence of at least one contaminant organism to provide an obtained genome sequence of at least one contaminant organism; and
calculating the frequency distribution of N-mers of the obtained genome sequence of at least one contaminant organism.

6. The method of claim 1, wherein determining frequency distribution of N-mers in the target polynucleotide is performed by:
obtaining the sequence of the target polynucleotide; and
calculating the frequency distribution of N-mers of the target polynucleotide.

7. The method of claim 1, wherein the selecting further comprises:
polymerizing nucleotides to provide primers having sequences of the selected of N-mers,
performing an amplification reaction with the primers with a sample comprising nucleic acid of the target organism,
detecting amplification, and
selecting N-mers of about 9 to 16 nucleotides for manufacturing oligonucleotide probes based on the detection.

8. The method of claim 7, wherein detecting amplification is performed by gel electrophoresis, sequencing, and/or RT-PCR based assays.

9. A method to select a set of N-mers of about 9 to 16 nucleotides for manufacturing oligonucleotide probes biased towards a target polynucleotide, the method comprising:
determining N-mers of the target polynucleotide to identify N-mers specific to the target polynucleotide;
determining a frequency distribution and location of the N-mers specific to the target polynucleotide within the target polynucleotide to provide a target N-mers distribution;
determining a frequency distribution of the N-mers specific to the target polynucleotide within one or more contaminants to provide a contaminant N-mers distribution; and
selecting a set N-mers based on the target N-mer distribution and location, and on the contaminant N-mers distribution to provide a selected N-mers for the manufacturing of the oligonucleotide probes.

10. A method of amplifying a target nucleic acid with termination, the method comprising:
providing a polymerase-target sample mixture comprising a polymerase, primers comprising sequences of biased N-mers selected by the method of claim 1, and a sample comprising a target nucleic acid and a contaminant nucleic acid; and
mixing dideoxynucleotides and deoxynucleotides to the polymerase-target sample mixture to allow amplification of the target nucleic acid thus producing an amplification mixture.

11. The method of claim 10, wherein the providing is performed by:
providing a primer-target sample mixture;
incubating the primer-target sample mixture under conditions to allow hybridization between the primers and the target nucleic acid in the primer-target sample mixture;
mixing a polymerase with the primer-target sample mixture to produce a polymerase-target sample mixture.

12. The method of claim 11, wherein providing a primer-target sample mixture is performed by:mixing primers comprising sequences of biased N-mers with a sample comprising a target nucleic acid and a contaminant nucleic acid, to produce a primer-target sample mixture.

13. The method of claim 11, wherein providing a primer-target sample mixture is performed by:
mixing primers comprising sequences of biased N-mers and random hexamers, with a sample comprising a target nucleic acid and a contaminant nucleic acid, to produce a primer-target sample mixture.

14. The method of claim 10, wherein the polymerase-target sample mixture further comprises a reverse transcription enzyme.

15. The method of claim 13, wherein the polymerase-target sample mixture further comprises a reverse transcription enzyme

16. The method of claim 10, wherein the polymerase is an isothermal polymerase.

17. The method of claim 16, wherein the polymerase is φ29 DNA polymerase.

18. The method of claim 10, wherein the polymerase is a mesophilic or thermophilic polymerase.

19. The method of claim 10, wherein mixing the dideoxynucleotides and deoxynucleotides further comprises
determining a length distribution of synthetic fragments,
calculating a ratio of dideoxynucleotides to deoxynucleotides based on the determining to identify a ratio associated with a desired synthetic fragment length to provide a calculated ratio, and
mixing dideoxynucleotides and deoxynucleotides in concentrations based on the calculated ratio.

20. The method of claim 10, wherein the primers comprise natural and modified bases.

21. The method of claim 20, wherein the natural and modified bases are phosphothioated bases.

22. A system for amplification of a target nucleic acid sequence in a sample comprising a target organism nucleic acid and a contaminant organism nucleic acid, the system comprising:
primers having sequences of N-mers, a polymerase, dideoxynucleotides, deoxynucleotides, and reagents for simultaneous combined or sequential use in the method of claim 10.

23. The system of claim 22, wherein the system is comprised in a portable point of care device or non-portable point of care device.

24. A manufactured oligonucleotide, obtained by polymerizing nucleotide to have sequences of N-mers selected from claim 1.

25. A computer-based method to select N-mers of about 9 to 16 nucleotides biased towards a target polynucleotide of a target whole organism, the method comprising the following computer-operated steps wherein a computer performs the steps in single-processor mode or multiple-processor mode:
determining frequency distribution and location of N-mers in the target polynucleotide to obtain a target N-mer distribution;
determining frequency distribution of N-mers in contaminants to obtain a contaminant N-mers distribution; and
selecting a set of N-mers based on the target N-mer distribution and location, and on the contaminant N-mers distribution and location to provide a selected N-mers for the manufacturing of the oligonucleotide probes.

26. A physical computer readable medium comprising computer executable software code stored in said medium, which computer executable software code, upon execution, carries out the method of claim 25.

27. A method of detecting a target nucleic acid, the method comprising
providing a polymerase-target sample mixture comprising a polymerase, primers comprising sequences of biased N-mers selected by the method of claim 1, and a sample comprising a target nucleic acid and a contaminant nucleic acid;
mixing dideoxynucleotides and deoxynucleotides to the polymerase-target sample mixture to allow amplification of the target nucleic acid thus producing an amplification mixture; and
contacting the amplification mixture with a probe suitable to detect the target nucleic acid.

28. A system for detection of a target nucleic acid in a sample comprising a target organism nucleic acid and a contaminant organism nucleic acid, the system comprising:
primers having sequences of N-mers, a polymerase, a probe for specific detection of the target polynucleotide, and reagents for simultaneous combined or sequential use in the method of claim 27.

29. A device to perform detection of a target polynucleotide, the device comprising:
a first component configured to perform amplification of one or more target nucleic acid from a sample to provide an amplification mixture,
a second component configured to perform specific detection of one or more target nucleic acid from the amplification mixture, and
an electronic interface to collect data from portable point of care device or non-portable point of care device to a computer or smart device.

30. The device of claim 29, further comprising a computer unit configured to select N-mers for manufacturing oligonucleotide primers suitable to be used in the amplification performed in the first component.
